(19) United States
(12) Patent Application Publication
(10) Pub. No.: US 2006/0154325 A1
(43) Pub. Date: Jul. 13, 2006

(54) Synthesis of epoxide based inhibitors of cysteine proteases

(76) Inventors: Matthew Bogyo, Redwood City, CA (US); Steven H.L. Verhelst, Stanford, CA (US)

Correspondence Address: PETERS VERNY JONES & SCHMITT, L.L.P., 425 SHERMAN AVENUE, SUITE 230, PALO ALTO, CA, 94306, US

(21) Appl. No.: 11/329,818

(22) Filed: Jan. 10, 2006

Related U.S. Application Data
(60) Provisional application No. 60/642,891, filed on Jan. 10, 2005.

(51) Int. Cl. C12Q 1/37; C07K 7/08; C07D 303/08

(52) U.S. Cl. 435/23; 530/402; 549/551

(57) ABSTRACT

Epoxide based cysteine protease inhibitors containing peptide derivatives and methods for synthesizing them in sold phase are disclosed. Preferably, an epoxy succinyl “warhead” (for binding to the enzyme) is prepared, having two amino acid residues or residue-like structures on either side. Natural and non-natural amino acids may be used. The present process may be carried out entirely on a solid support, with the proviso that a dipeptide like group is prepared prior to coupling to one side of the supported complex. The method lends itself to more efficient inhibitor synthesis, and may be employed with mixtures of peptide compounds, and various modifications of epoxides to create diverse libraries of inhibitors.

### CROSS-REFERENCE TO RELATED APPLICATIONS

This application claims priority to provisional patent application Ser. No. 60/642,891, filed Jan. 10,2005, entitled “Synthesis of Epoxide Based Inhibitors of Cysteine Protease,” and hereby incorporated by reference.

### STATEMENT OF GOVERNMENTAL SUPPORT

This invention was made during work supported by U.S. National Institutes of Health under grant U54 RR020843. The government has certain rights in this invention.

### REFERENCE TO SEQUENCE LISTING OR COMPACT DISK

None.

### BACKGROUND OF THE INVENTION

1. Field of the Invention

The present invention relates to the synthesis of organic molecules useful in the labeling or inhibition of cysteine proteases.

2. Related Art

Cysteine proteases are proteolytic enzymes, which utilize a cysteine residue for their catalytic activity. They can be grouped in at least 30 protein families. Each family contains proteins with similar amino acid sequences and evolutionarily conserved sequence motifs, which are reflected in the family members' similar 3D structures.

Proteases in Family C1 (the papain family) include mammalian enzymes such as cathepsins B and L, which are thought to be involved in cancer growth and metastasis. Cathepsin K is considered to be involved in bone degradation an osteoporosis. Family C1 also includes parasitic enzymes being essential for the parasite-host interaction (e.g. cruzipain from Trypanosoma cruzi—ausing Chagas' disease, and falcipain from Plasmodium falciparum—causing malaria).

Enzymes belonging to Family C13 (the legumain family) have been shown to play key roles in antigen presentation. Interleukin converting enzyme (ICE) and other enzymes belonging to Family C14 (the caspase family) have gained much interest recently, as key mediators of apoptosis. Many proteases of pathogenic bacteria are virulence factors and cause severe problems for the host at infections, such as gingipains (Family C25) of Porhyromonas gingivalis in periodontitis and streptopain (Family C10) from Streptococcus pyogenes.

Proteolysis controls a number of essential biological processes ranging from cell division to cell death. Amongst the cysteine proteases, the lysosomal cathepsins play important roles in a number of human diseases. [Ref. 6] However, this family displays similar substrate preferences making the development of tools to study individual cathepsins a challenging task.

Joyce et al. (Cancer Cell, 2004, 5, 443-453) have shown the importance of the cysteine cathepsin proteases in a number of processes related to cancer progression. These processes include tumor growth, angiogenesis, and metastasis. This article discloses that a general, covalent inhibitor, which specifically targets the cathepsin protease family can be administered systemically to transgenic mice that develop a multi-stage form of pancreatic cancer and lead to reduced tumor burden, metastasis and angiogenesis.

Specifically, Joyce et al. disclose the inhibition of six cathepsin proteases by compounds DCG-04, 530/550 Biodipy DCG-04, and JPM-OEt. These general covalent epoxide based inhibitors showed little or no general toxicity when administered to normal mice over a period of several weeks. This work (including work by the present inventors) suggests that cathepsin cysteine proteases represent a valid target for anti-cancer therapeutics.

The general epoxide scaffold for JPM-OEt is based on the natural product E-64, which was discovered to be a natural product inhibitor of cysteine protease in 1978 (Hanada, K. et al. Agric. Biol. Chem. 1978 42, 523-528 and 529-536).

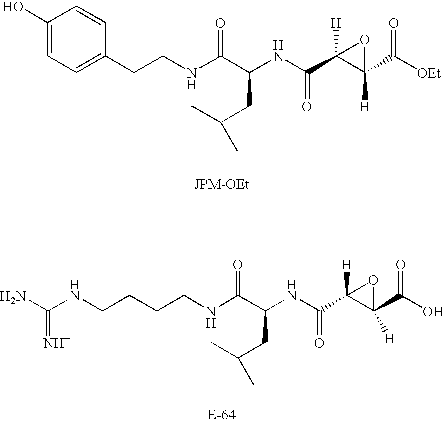

The related compound JPM-565 is identical to JPM-OEt except that the ethyl ester is converted to the free carboxylic acid.

These classes of compounds (i.e. having a succinyl epoxy component) have been used as cysteine protease inhibitors since 1978. A number of research groups have synthesized analogs of the general epoxide structure over the past 20+ years and the crystal structure of E-64 bound to various cysteine proteases in the cathepsin family were reported as early as 1989.

Meara and Rich, J. Med. Chem. 1996, 39, 3357-3366 outline some basic SAR data on the epoxysuccinyl inhibitors of cysteine cathepsins. They worked with the basic epoxy succinyl structure

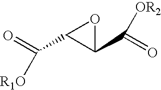

They prepared molecules where OR₂ was replaced by a substituted carboxamides, e.g. where the OR₂ was replaced with NH—CH(C₄H₉)—C(O)—NH—C₅H₉.

The use of JPM-565, which is the free acid version of JPM-OEt, is disclosed in Shi, et al. J. Biol. Chem. 1992, 267, 7258-7262. This paper discloses the molecular cloning and expression of human alveolar macrophage cathepsin S, and its active site labeling with JPM-565. Other inhibitors, such as E-64, cystatin c, Cbz-TYr-Tyr(o-butyl)-CHN₂ are also disclosed.

Greenbaum et al. Chem. Biol. 2002, 9, 1085-1094 also describe cysteine protease inhibitors. It is noted there that the epoxide class of cysteine protease binding compounds has a chiral structure, e.g. as shown. The following general inhibitor is disclosed:

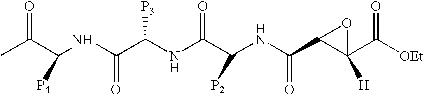

Libraries of different amino acids substituted for P2, P3, and P4 were prepared. The libraries were first prepared by fixing each of the P2, P3, and P4 positions with each of the 20 possible natural amino acids (minus cysteine and methionine, plus norleucine). A mixture of the same natural amino acids was used in the remaining two amino acid positions, resulting in 19 P2, P3 and P4 sublibraries, with each made up of a mixture of 361 compounds (19×19). The three sets of sublibraries (each set containing a representative of each amino acid in the position of interest) were assayed against purified protease targets by competition with the radiolabelled active site-directed probe ¹²⁵I-DCG-04. Competition, i.e. loss of labeling, was indicative of inhibition by the unlabelled library member. For example, in FIG. 2 of the paper it can be seen that cathepsin K was most inhibited by Val, Ile or Leu in the P2 position. Both S,S and R,R enantiomer libraries were prepared.

However, no conclusion can be drawn regarding any specific inhibitor with, e.g. Val in the P2 position, since the performance of that embodiment was only measured with a mixture of amino acids in the P3 and P4 positions.

The small molecules that label subsets of enzymatic proteins have been developed as a means to simplify complex proteomes and allow bulk profiling of enzyme activity. [Ref. 2] These activity based probes (ABPs) combine tags for visualization or purification with warheads that covalently attach to the active sites of enzymatic targets in an activity-dependant manner. Using this approach, a specific protein or protein family can be readily monitored in complex protein mixtures, intact cells, and even in vivo. [Ref. 3] Furthermore, enzyme class specific probes can be used to develop screens for small molecule inhibitors that can be used functional studies.[Ref. 4,5] Therefore, methods that will facilitate the development of novel ABPs have great value for advancing the use of this technology. The natural product E-64 [Ref. 1] inhibits cysteine proteases via covalent attachment to the active site sulfhydryl nucleophile. This reagent contains a leucine residue that mimics the critical P2 residue of a substrate and therefore binds efficiently in the S2 specificity pocket of virtually all cysteine cathepsins. As a result this reagent is a broad-spectrum inhibitor and activity probe.

One of the first ABPs reported using the epoxide warhead, DCG-04 [Ref. 2], has found widespread use for a number of functional studies of the cysteine cathepsins. However, this general probe makes use of a single peptide piece that makes contacts with only one side of the protease active site. A recent crystal structure of a double-headed epoxide inhibitor showed that the entire structure binds along the active site cleft with contact made on both sides of the reactive cysteine nucleophile. [Ref. 8] Described below is the development of a method that would allow facile attachment of diverse peptide sequences on both sides of the epoxide warhead, rather than only one, in the solid phase. This is intended, among other things, to facilitate the synthesis of probes with increased selectivity compared to the general DCG-04 probe.

### BRIEF SUMMARY OF THE INVENTION

The present invention is directed to epoxy-succinyl- peptide- and peptoid- like inhibitors and solid phase methods for their synthesis. The invention contemplates novel compounds, as well as novel methods for generating known compounds and libraries of compounds. The solid phase methods of synthesis also permit the creation of a diverse library of compounds by adding different peptide derivatives to different resins as is known in the art of peptide synthesis. This includes the addition of both peptide and non-peptidic elements on both sides of the central epoxy-succinyl reactive functional group.

Methods of synthesis and use are also comprised in the present invention. The compounds that may be synthesized by the present solid phase synthetic method and that may be expected to have activity in the inhibition of cysteine proteases may include several different, novel structures.

They may be represented as COMPOUND 1 and COMPOUND 2: 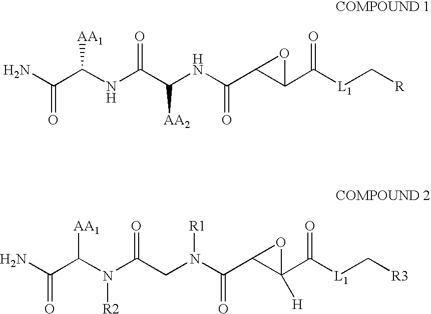
where AA₁ and AA₂ represent amino acid side chains, preferably hydrophilic amino acid side chains (in increasing order of hydrophilicity: Gly [i.e. no side chain], Thr, Ser, Trp, Tyr, Pro, His, Glu, Gln, Asp, Asn, Lys, Arg); L₁ is NH or O, preferably with the proviso that if L₁ is NH, R may be a peptide, preferably a dipeptide, and preferably having a natural peptidyl backbone; R1 and R2 may independently any of the twenty naturally occurring natural amino acid side chains, and R and R3 are peptide or peptoid moieties, respectively, preferably having two amino acids, and preferably being peptide in COMPOUND 1 and peptoid, as illustrated in COMPOUND 2. COMPOUND 1 and 2 may be either R,R or S,S enantiomers.

COMPOUND 2 represents a peptoid embodiment of the present invention, and the AA₁ side chain will only be present in hybrid peptide/peptoid embodiments.

In addition, the present synthetic methods may be used to created epoxysuccinyl compounds having various organic linkers and substitutions, as illustrated by compounds 7-12 in FIG. 3. In this case, the illustration of COMPOUND 1 would be varied at * and R₄ as shown in COMPOUND 3:

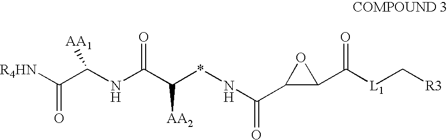

COMPOUND 3 is synthesized in similar fashion to COMPOUND, except that additional linkers may be inserted at *, and R4 may be other than H, i.e. a lower alkyl diamide. An example of R₄ is given in compounds 7, 8 and 11, namely

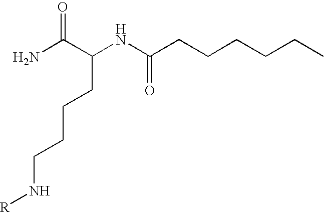

Other lower alkyl diamide or peptide-like compounds may be readily designed, given the present teachings, for use in the present solid phase synthesis method. The linker * is preferably between 0 and 10, most preferably 0-4, with 4 being illustrated in compounds 9, 10 and 12. Examples of COMPOUND 2 tested with rat liver extracts are L1=O, R=OH or CH3, and AA1=H.

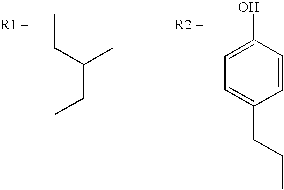

### BRIEF DESCRIPTION OF THE DRAWINGS

FIG. 1 is a schematic diagram of a generalized method of synthesis of the present peptide-related compounds;

FIG. 2 is a schematic diagram of a generalized method of synthesis of the present peptoid-related compounds;

FIG. 3 (top) is a representation of several compounds discussed herein; and (bottom) a representative synthesis of an epoxysuccinyl-based cysteine protease inhibitor having two peptide derivatives flanking the epoxysuccinyl “warhead”;

FIG. 4 is a representation of gels obtained form reactions of several cysteine proteases with compounds according to the present invention; Left panel: direct labeling of rat liver homogenates with radiolabeled Ac-SV4 and Ac-SV5. Right panel: direct labeling using fluorophore labeled TR-SV4 and TR-SV5. Δ=preheat; and

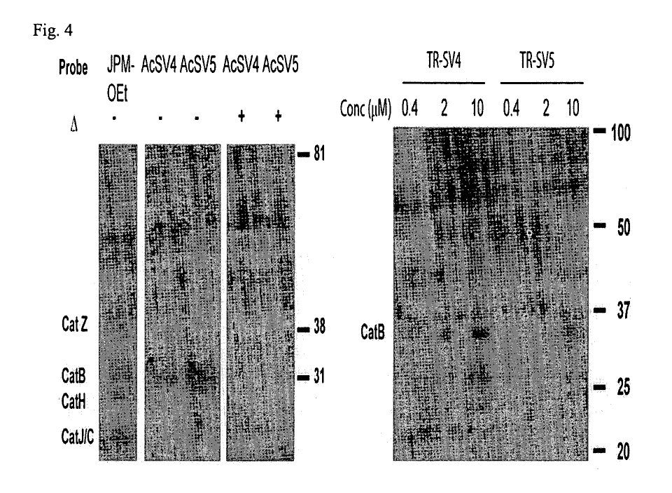

FIG. 5 is a representation of gels showing competition experiments in rat liver using probes based on compounds according to the present invention. Competition experiments in rat liver homogenates used increasing concentrations of probe (lane 1 to 9: 0 nM, 0.6 nM, 3.2 nM, 16 nM, 80 nM, 400 nM, 2 μM, 10 μM and 50 μM, respectively). Radiolabeled general cysteine protease ABP JPM-OEt was added to monitor remaining cysteine protease activity.

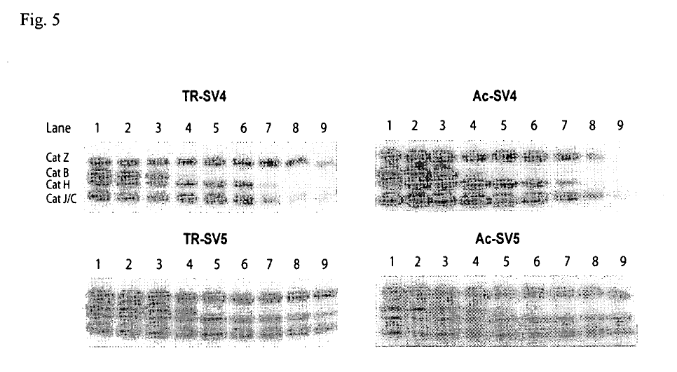

### DETAILED DESCRIPTION OF THE PREFERRED EMBODIMENT

Definitions

“Peptide derivative” means a compound comprising an oligopeptide that may have modifications to peptide side chains and/or organic groups serving as linkers or spacers inserted between amino acids. The term “peptide derivative” may be further understood by reference to the illustrative embodiments in the accompanying description and figures, e.g. compounds 7-12, and COMPOUNDS 1-3, although the term is not limited to these examples. The term “peptide derivative,” in the context of the present methods, includes known compounds as illustrated herein. The present peptides are preferably 1-5, preferably 1-3 amino acids, and will include various modifications and derivatives of naturally occurring amino acids, as well as non-natural amino acids. As is known, the naturally occurring amino acids are: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Particularly preferred are leucine and tyrosine, particularly leucine as AA2.

Thus, the term “amino acid derivative,” such as may be a residue in a peptide derivative, includes, in addition to the modifications specifically illustrated below, derivatives such as -tyrosine methyl ester, L-3,5-diiodotyrosine methyl ester, L-3-iodotyrosine methyl ester, β-(4-hydroxy-naphth-1-yl)-L-alanine methyl ester, β-(6-hydroxy-naphth-2-yl)-L-alanine methyl ester, and the like, as are described in U.S. Pat. No. 6,949,570 to Ashwell et al. hereby incorporated by reference for purposes of describing the synthesis, structure and use of certain amino acid derivatives. Other examples of amino acid derivatives that may be utilized in the present methods and compounds are given in U.S. Pat. No. 6,900,196 to Liebeschuetz, et al. entitled “Serine protease inhibitors,” U.S. Pat. No. 6,133,461 to Inaba, entitled “Process for producing amide derivatives and intermediates therefore,” and U.S. Pat. No. 5,346,907 to Kerwin, Jr. et al., entitled “Amino acid analog CCK antagonists,” all of which are hereby incorporated by reference in their entirety as describing exemplary amino acid analogs, modifications and derivative useable in the present solid phase synthetic method.

The term “Peptide” is used in its scientifically accepted sense to mean any compound produced by amide formation between a carboxyl group of one amino acid and an amino group of another. The amide bonds in peptides may be called peptide bonds. The word peptide usually applies to compounds whose amide bonds are formed between C-1 of one amino acid and N-2 of another (sometimes called eupeptide bonds), but it includes compounds with residues linked by other amide bonds (sometimes called isopeptide bonds).

The term “Epoxide” is used in its scientifically accepted sense to mean an organic ether which the oxygen atom is part of a ring of three atoms. As used herein, an epoxide may contain additional N, O, S or C atoms.

Accordingly, “diester epoxide” means an epoxide as described above with esters groups attached directly to each of the carbon atoms in the epoxide ring. The ester can be any type of ester not simply an ethyl or nitrophenyl ester.

“Protective group” or “protecting group” is used in its scientifically accepted sense to mean the group, which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive oxygen atoms present in the reactants. Acceptable protective groups for alcoholic or phenolic hydroxyl groups, which may be removed successively and selectively includes groups protected as acetates, haloalkyl carbonates, benzyl ethers, alkylsilyl ethers, heterocyclyl ethers, and methyl or alkyl ethers, and the like. Protective or blocking groups for carboxyl groups are similar to those described for hydroxyl groups, preferably tert-butyl, benzyl or methyl esters. Examples of protecting groups can be found in T. W. Greene et al., Protective Groups in Organic Chemistry, (J. Wiley, 2.sup.nd ed. 1991) and Harrison et al., Compendium of Synthetic Organic Methods, Vols. 1-8 (J. Wiley and Sons 1971-1996).

“Amino-protecting group” is used in its scientifically accepted sense to mean a protecting group intended to protect a nitrogen atom against undesirable reactions during synthetic procedures and includes, but is not limited to, benzyl, benzyloxycarbonyl (carbobenzyloxy, CBZ), p-methoxybenzyloxycarbonyl, p-nitrobenzyloxycarbonyl, tert-butoxycarbonyl (BOC), trifluoroacetyl, and the like. It is preferred to use FMOC as the amino-protecting group because of the relative ease of removal using mild bases such as piperidine. Also this protecting group allows linkage of peptides to the resin though bonds that can be cleaved by simple tri-fluoroacetic acid treatment; or by catalytic hydrogenation in the case of CBZ.

“Deprotection” or “deprotecting” is used in its scientifically accepted sense to mean the process by which a protective group is removed after the selective reaction is completed. Certain protective groups may be preferred over others due to their convenience or relative ease of removal. Deprotecting reagents for protected hydroxyl or carboxyl groups include potassium or sodium carbonates, lithium hydroxide in alcoholic solutions, zinc in methanol, acetic acid, trifluoroacetic acid, palladium catalysts, or boron tribromide, and the like.

“Rink Resin” is used in its scientifically accepted sense to mean an amide-releasing, acid-cleavable solid support, for example 4-(2′,4′-Dimethoxyphenylaminomethyl)phenoxy polystyrene with an Fmoc protective group. A suitable Rink resin is a PEG-PS resin prepared from aminomethyl copoly(styrene-1% DVB), 100-200 mesh. This resin is prepared by derivatization of aminomethyl NovaGel™ with the modified Rink linker. NovaGel™ resins combine the high functionality of polystyrene resins with the excellent swelling properties of PEG-PS type supports. This resin is sold under license from Sanofi Aventis, European patent 322,348 and US patent 5,124,478.

“Solid phase support,” or “solid support,” means any support or carrier capable of binding peptide derivatives. Well-known supports or carriers include glass, polystyrene, polypropylene, polyethylene, dextran, nylon, natural and modified celluloses, polyacrylamides, gabbros and magnetite. The nature of the carrier can be either soluble to some extent or insoluble for the purposes of the present invention. The support material may have virtually any possible structural configuration so long as the coupled molecule is capable of binding an amino acid group. Alternatively, the surface may be flat such as a sheet, test strip, etc. Preferred supports or carriers include polystyrene beads.

Epoxysuccinyl Compounds

The compounds of the present invention may exist in different chiralities. Unless indicated otherwise, both chiralities are intended by a given structure that does not show up or down bonds.

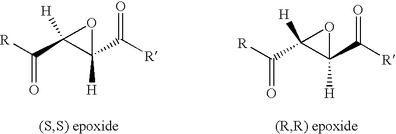

The formulas above illustrate the structure of the general epoxysuccinyl group. The chiral molecule can be synthesized in the 2R, 3R or 2S, 3S configuration depending on the starting material used. In this structure the R group represents the peptide portion that binds in the non-prime side of the enzyme active site and R′ represents the peptide portion that binds in the prime side of the active site. In general inhibitors of this class may flip in the active site to display amino acids into pockets on both sides of the active site. For the compound JPM-OEt the Leucine-tyramine portion of the molecule is attached at the R site. The R′ is a free acid (for JPM-565) or ester (for JPM-OEt). For both JPM compounds the S,S enantiomer is used.

In the compounds described below, it is understood that both enantiomers of the epoxysuccinyl group (2R,3R) and (2S,3S) can be linked to peptides composed of amino acids or amino acid derivatives to generate inhibitors. In general, it is possible to generate more selective inhibitors using the R,R enantiomer, but that the compounds tend to be less potent. However, S,S epoxysuccinyl peptide compounds may also yield selective inhibitors.

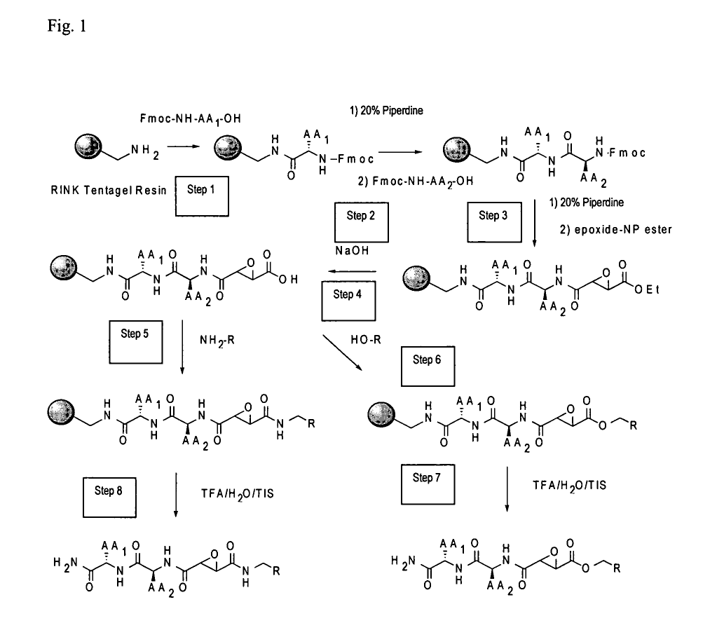

Generalized Methods: FIG. 1

A generalized method of synthesis of the present compounds is represented in FIG. 1, which shows a solid phase synthesis of “double headed” inhibitors based on the epoxysuccinyl reactive group. As shown in FIG. 1, an amino acid moiety is first attached to the solid support through its carboxy terminus. Additional amino acids, as desired, are added by conventional synthetic chemistry. Then in step 3, the epoxide group is added as a nitrophenolic ester. After hydrolysis of the ethyl ester on the resin, the resulting epoxy acid can be either coupled with amines (step 8) to yield amides or with alcohols (step 7) to yield esters. The alcohols yield pro-drugs that are likely to be processed to the active free acids inside cells. In both cases the R group will be added to increase the solubility, and membrane permeability. The R group is therefore selected from the list of groups set forth herein. It can range from amino acids to any of a variety of chiral or achiral amine and alcohols.

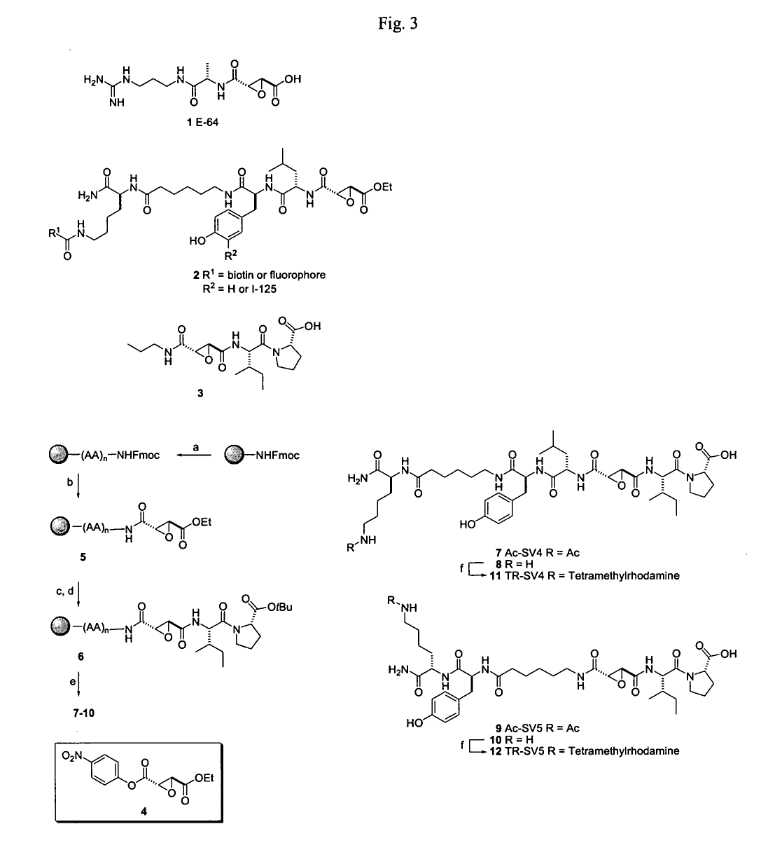

Using the method illustrated in FIG. 1, one may synthesize a variety of analogs of JPM-OEt in which the tyramine is replaced with other more water-soluble groups. In step 1, an FMOC protected amino acid residue is coupled to an amino-coupled resin. In step 2, a second amino acid group is then coupled to the immobilized compound, yielding AA₁-AA₂ —protective group (Fmoc). In step 3, the epoxide—nitro phenyl ester is added. This compound is illustrated as compound 4 in FIG. 3, and is an activated nitrophenyl ester. In step 4, the ester is hydrolysed to the corresponding acid with a base. In step 5, an amine (NH₂—R) is added to the acid group. R is preferably a peptide derivative synthesized prior to coupling, such as Ile-Pro, illustrated in FIG. 3, but R may also comprise non peptidyl components, which may be coupled by known means. In the present scheme, PyBOP (Benzotriazole-1-yl-oxy-tris-pyrrolidino-phosphonium hexafluorophosphate) is used as a coupling reagent.

In step 8, of FIG. 1, the compound of step 5 is cleaved from the solid support. In an alternative embodiment, an alcohol (HO—R) is used in place of the amine in step 5. This is illustrated in step 6. This results in an epoxy-succinyl ester, rather than an amide, and is also cleaved from the resin, as is illustrated in step 7.

This synthesis yields a range of double headed compounds and test for selectivity and potency across a range of cathepsin targets. Any compounds that seem promising in terms of potency, solubility and selectivity are tested in the RIP-Tag model, or other suitable animal model generally accepted in the scientific community as predictive of cysteine protease effects, especially tumorigenesis. In the RIP-Tag model, an oncogene, SV40 large T antigen (Tag), is expressed in islet beta cells under the control of the rat insulin promoter, Ripi . Several lines of Rip 1-Tag mice have been generated and characterized and are available to researchers.

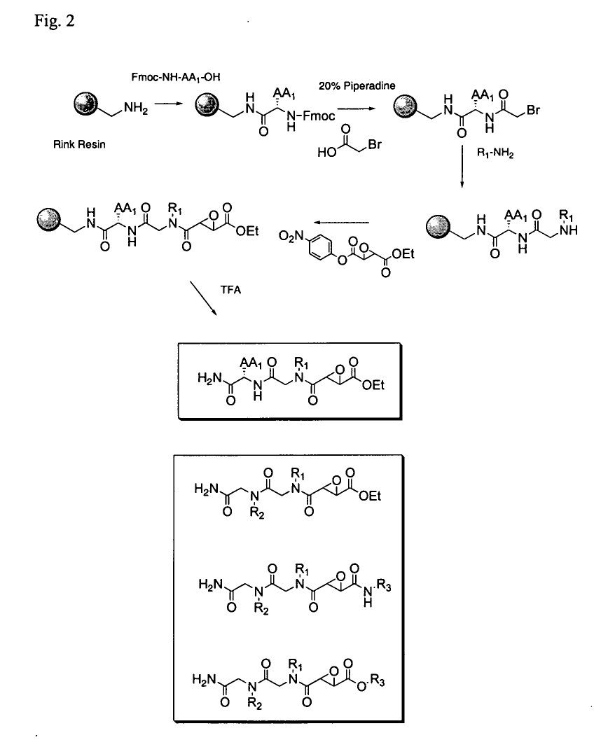

Peptoid Synthesis: FIG. 2

FIG. 2 illustrates the synthesis of peptoid inhibitors such as COMPOUND 2. FIG. 2 represents a series of synthetic reactions, that can be described, step by step, as follows:

In the first step, an amino Rink resin is deprotected to expose a free amine. In the second step, a protected amino acid (AA₁) is coupled to the resin. In the third step, a bromo acetic acid is coupled to the amino acid. In the fourth step, an R1-NH2 group is added, where R1 may be an aliphatic, aromatic, basic or acidic residue. Typically, amines are used that contain functional groups that mimic the sidechain groups of natural amino acids (i.e. carboxylic acids, amides, phenyl and aromatic groups, alkyl groups, hydroxyls, thiols, and imidazoles). More specific groups useful as R1 ands R2 are those found in the published structures of the naturally occurring amino acids. Fourthly, the epoxy succinyl warhead is coupled using the activated nitro phenyl ester. The compound synthesized in solid phase may then be eluted from the Rink resin with TFA (trifluoroacetic acid). Other eluting agents, such as HOAc, may be found suitable for this step.

In this way, peptoids with selected amino acid-like side chains can be prepared at positions R1 and R2. Epoxides can also be used that contain diverse R3 groups. This R3 group can be any number of amines or alcohols that contain additional functional groups such as amine, carboxylic acids, amides, phenyl and aromatic rings, heterocyclyic rings, and imidazoles.

In addition, as shown in the box in FIG. 2, R3 can be linked through either an NH or O group, as in the case with the peptide, there have been prepared several peptoid derivatives that are improvements on DCG-04 and also JPM-OEt.

Compounds shown in FIG. 2 were tested in total rat liver extracts, with the results that the peptoid analogs are active against the cathepsin targets, although the compounds tested were slightly less potent that the peptide embodiment described above. The method of FIG. 2 enables the synthesis of peptoids and peptoid/peptide hybrid epoxides. These general scaffolds can be achieved by solid phase synthesis of the main peptide or peptoid piece followed by attachment of the reactive epoxide. The resulting products can be cleaved from the resin using TFA. The structures at the bottom represent additional peptoids that can be synthesized using this method.

While the peptoids are slightly less potent that the peptide counterparts, they are nonetheless active and show favorable selectivity profiles. They also are likely to be more physiologically more stable and have better pharmacodynamic properties. Also, they can be optimized to increase potency by variations in the R3, R3 and peptide portions of the compounds.

Improved Solid Phase Synthesis: FIG. 3

The present invention further concerns an improved solid phase synthesis of cathepsin B specific ABPs, including the compounds described above. In the past, selective inhibitors for both cathepsin B and L have been reported. Most of these compounds, such as CA-074 (3) [Ref. 9], have taken advantage of diversity elements on both sides of the epoxide. In fact, virtually all of the selectivity for cathepsin B has been derived by specific interactions with residues on the occluding loop found only in this protease. This unique specific interaction has been used to generate small molecules that target cathepsin B. However, the previously reported inhibitors and probes were obtained by time consuming solution phase synthesis [Ref. 10], thus limiting the extent of structure activity studies that could be performed.

In order to efficiently synthesize double-headed epoxysuccinyl probes there is a need for a solid phase synthesis protocol that would allow peptide synthesis to be carried out followed by attachment of the epoxide group and then further elongation of the probe.

The initial probe design (described below) was based on the broad spectrum ABP DCG-04 [Ref. 3] containing the dipeptide of CA-074 [Ref. 3]. The upper portion of FIG. 3 shows compounds E-64, a known peptide-like scaffold; DCG 04, compound 2, an activity based probe having a biotin, fluorophore or radioactive label; and compound 3, the above-referenced CA-074. The lower portion of FIG. 3 outlines the basic strategy, which makes use of standard solid phase peptide chemistry, followed by capping of the N-terminus with the epoxysuccinyl synthon using the activated nitrophenyl ester 4 [Ref. 11 ]. The epoxysuccinyl ester 5 can then be hydrolyzed on resin thereby allowing elongation of the probe on the other side of the warhead. The use of a PEG-based Rink-resin permitted the use of alcohols as solvent system during this ester bond cleavage. Thus, hydrolysis of ethyl ester 5 was achieved by treatment with a 0.25 M KOH solution in ethanol. LCMS analysis after test cleavage showed that clean hydrolysis was complete in approximately 20 min. Additionally, polystyrene-based resins may be used with a solvent system of alcohol and THF rather than the aqueous KOH solutions used for PEG based resins. Various alcohol solvents may be used, e.g. methanol, ethanol, propanol, and butanol.

Since elongation at the distal side of the epoxide takes place in the opposite direction of standard solid phase peptide synthesis, it was necessary to minimize the couplings performed after ester hydrolysis. Therefore, protected Fmoc-Ile-Pro-OtBu was synthesized in solution and Fmoc-deprotected prior to coupling to the free acid on solid support. This coupling was achieved using PyBOP as a coupling reagent.

Using the above-described protocol, compounds 7-10 were obtained in 20-36% yield after HPLC purification. Free amines 8 and 10 were conjugated to a tetramethylrhodamine (TR) fluorophore. Probes were synthesized containing a leucine in the P-2 position (Ac-SV4 7 and TR-SV4 11) as well lacking the leucine residue (Ac-SV5 9 and TR-SV5 12).

The potency and selectivity of the probes were determined by labeling of cathepsin cysteine proteases in crude rat liver homogenates. Labeling with I-125-labeled versions of Ac-SV4 and AC-SV5 indicated that Ac-SV5 is highly specific for cathepsin B, whereas Ac-SV4, while somewhat selective for cathepsin B, at high concentrations targets all the primary active cathepsins (B, Z, H, J and C; FIG. 2). Preheat controls confirm that labeling is activity dependent. Correspondingly, the fluorophore containing probes show similar potency and specificity patterns, however with a slightly higher non-specific labeling of other proteins.

To quantify the potency of the SV4 and SV5 probes, competition experiments were performed using the general cysteine protease ABP JPM-OEt.[Ref. 12] Thus, after addition of the double headed ABPs over a range of concentrations, radiolabeled JPM-OEt was added to monitor the remaining activity of the cathepsins in the rat liver homogenates. Both the SV4 and SV5 compounds have a defined preference for cathepsin B.

The competition data from FIG. 3 were quantified using image analysis software and used to determine the concentrations at which 50% of the enzyme activity was inhibited (apparent IC50 values; see Table 1).

TABLE 1
Apparent IC50 values of ABPs for different
cathepsins in rat-liver homogenates.

| | IC50 | IC50 | IC50 | IC50 | Cat B |
|---|---|---|---|---|---|
| ABP | Cat B | Cat Z | Cat H | Cat J/C | selectivity |
| Ac-SV4 | 8 | 15 × 10³ | 1.2 × 10³ | 4.0 × 10³ | 1.5 × 10² |
| TR-SV4 | 6.8 | 11 × 10³ | 1.1 × 10³ | 1.5 × 10³ | 1.6 × 10² |
| Ac-SV5 | 22 | >50 × 10³ | >50 × 10³ | >50 × 10³ | >2.3 × 10³ |
| TR-SV5 | 15 | >50 × 10³ | >50 × 10³ | >50 × 10³ | >3.3 × 10³ |

The SV4 probes have an IC50 values for cathepsin B of 7-8 nM making them slightly more potent than the SV5 probes, which showed IC50 values of 15-22 nM. However, the loss in potency is accompanied by a 10-fold increase in selectivity for cathepsin B relative to the SV4 series of probes. Thus, the SV5 probes were greater than 2300 times more reactive towards cathepsin B than the other predominant cathepsins.

The selectivity of the majority of the previously published cathepsin B-specific inhibitors is derived from distinct interactions with the unique occluding loop on cathepsin B (Cys 108-Cys 119). Crystal structures of enzyme-inhibitor complexes have shown that the isoleucine-proline dipeptide portion of compounds such as CA-074 bind such that the free carboxylate projects into the so-called S′ region of the active site. For the dipeptidyl peptidase cathepsin B a loop structure protrudes into this region and makes direct contacts with the free carboxylate through hydrogen bonds to two conserved histidine residues.[Ref. 13] While these contacts are what drive the primary specificity for cathepsin B, the interaction of a leucine residue in the hydrophobic P2 pocket is optimal for virtually all of the papain-fold cysteine proteases. Thus, the higher selectivity of the SV5 probes is most likely due to the loss of the hydrophobic leucine residue, thereby eliminating the general high affinity interactions with the P2 pockets of other cathepsins. This hypothesis also explains the slight reduction in overall inhibitor potency observed for the SV5 probes. This general paradigm suggests that by optimization of both the P2 binding element and the S′ binding elements it should be possible to generate higher selectivity of probes for individual members of this protease family.

It will be apparent to those skilled in the art that the present sold phase synthesis method may be adapted to combinatorial techniques. That is, a number of beads or pins may be reacted in parallel with different derivatives, and capped with different distal molecules. That is, the first peptide derivative and the second peptide derivative may be part of a mixture of different peptide derivatives, each attached to a solid support, for generating a plurality of different epoxy peptide derivatives. The supports, i.e. beads, are mixed together and simultaneously reacted with the desired epoxysuccinyl synthon ester, which is then cleaved to the acid on the solid support, and coupled to the distal moiety, or a mixture of distal moieties (R in FIG. 1).

Experimental

Synthesis of probes. After Rink amide Novagel (NovaBiochem) was loaded with the first Fmoc-protected amino acid, elongation took place by standard solid phase peptide chemistry, using 20% piperidine to cleave Fmoc-protecting groups and a combination of DIC (3 eq.) and HOBt (3 eq.) to condensate each amino acid (3 eq.). After final Fmoc deprotection, nitrophenyl ester 4 (3 eq.) in DMF was added to the resin and reacted for 1 h to cap the terminal amine functionality. Next, the ethyl ester was saponified using 0.25 M KOH in EtOH for approximately 20 min, and resin was subsequently washed with 1% AcOH in EtOH, EtOH and DCM. Finally, H-Ile-Pro-OtBu (3 eq.), was coupled to the free carboxylic acid under influence of PyBOP (3 eq.) and DIEA (6 eq.) in DMF. Deprotection of the probes and concomitant cleavage from the solid support was effected by TFA/H2O/TIS (95/2.5/2.5). Probes were precipitated with ether, collected by centrifugation and purified by HPLC.

Evaluation in proteomes. Rat liver homogenates were used at 1 mg/mL in 50 μL reaction volume in reaction buffer of pH 5.5 (50 mM sodium acetate, 2 mM DTT, 5 mM MgCl2). Controls were preheated for 5 min at 90° C. Samples were incubated for 0.5 h with either radiolabeled (106 cpm) or fluorophoreconjugated probes, and analyzed by SDS-gel electrophoresis. For competition experiments, ABPs were added to rat liver homogenates at the indicated concentrations and incubated at room temperature for 0.5 h. Subsequently, samples were treated with radiolabeled JPM-OEt (106 cpm) for an additional 0.5h prior to subjection to gel-electrophoresis. Data were quantified using NIH ImageJ and analyzed with GraphPad Prism. (San Diego, Ca.).

Summary

In summary, there is described a solid phase method for the synthesis of double headed epoxide inhibitors of cysteine proteases. One feature of this method includes the on-resin hydrolysis of the epoxysuccinyl ethyl ester and subsequent coupling of diversity elements while the compound is still on the resin.

Using this method, we show the efficient synthesis of two different classes of activity-based probes carrying either a radioactive or a fluorescent tag. Studies of these probes in complex proteomes show that removal of the hydrophobic P2 residue increases selectivity for cathepsin B, resulting in a novel, highly selective cathepsin B label. Furthermore, this method will facilitate the synthesis of additional probe families with range of diversity elements on both sides of the reactive warhead that are likely to yield additional protease specific reagents.

The present solid phase synthetic methods are applicable to a wide variety of dipetidyl epoxysuccinyl protease inhibitors. Novel compounds are disclosed and shown to have activity as cysteine protease inhibitors. These compounds share a motif of peptide-epoxysuccinyl-peptide.

The present examples, methods, procedures, specific compounds and molecules are meant to exemplify and illustrate the invention and should in no way be seen as limiting the scope of the invention, which is defined by the literal and equivalent scope of the appended claims. That is, given the teachings of the present specification, numerous variations of the illustrated embodiment may be created. Any patents or publications mentioned in this specification are indicative of levels of those skilled in the art to which the patent pertains and are intended to convey details of the invention which may not be explicitly set out but would be understood by workers in the field. Such patents or publications are hereby incorporated by reference to the same extent as if each was specifically and individually incorporated by reference and for the purpose of describing and enabling the method or material referred to.

References and Notes

[1] For example, see: (a) Tyers, M.; Mann, M. Nature 2003,422, 193-197. (b) Miklos, G. L. G.; Maleszka, R. Proteomics 2001, 1, 169-178. (c) Patterson, S. D.; Aebersold, R. H. Nature genetics 2003, 33, 311-323.

[2] Some recent reviews: (a) Campbell, D. A.; Szardenings, A. K. Curr. Opin. Chem. Biol. 2003, 7, 296-303. (b) Kozarich, J. W. Curr. Opin. Chem. Biol. 2003, 7, 78-83. (c) Jeffery, D. A.; Bogyo, M. Curr. Opin. Biotech. 2003, 14, 87-95. (d) Speers, A. E.; Cravatt, B. F. ChemBioChem. 2004, 5, 41-47.

[3] For example, see: Joyce, J. A.; Baruch, A.; Chehade, K.; Meyer-Morse, N.; Giraudo, E.; Tsai, F.-Y.; Greenbaum, D. C.; Hager, J. H.; Bogyo, M.; Hanahan, D. Cancer Cell 2004, 5, 443-453.

[4] Greenbaum, D. C.; Arnold, W. D.; Lu, F.; Hayrapetian, L.; Baruch, A.; Krumine, J.; Toba, S.; Chehade, K.; Broemme, D.; Kuntz, I. D.; Bogyo, M. Chem. Biol. 2002, 9, 1085-1094.

[5] Greenbaum, D.; Baruch, A.; Hayrapetian, L.; Darula, Z.; Burlingame, A.; Medzihradszky, K. F.; Bogyo, M. Mol. Cel.Proteomics 2002, 1, 60-68.

[6] Lecaille, F.; Kaleta, J.; Bromme, D. Chem. Rev. 2002,102, 4459-4488.

[7] Greenbaum, D.; Medzihradszky, K. F.; Burlingame, A.;Bogyo, M. Chem. Biol. 2000, 7, 569-581.

[8] Stern, I.; Schaschke, N.; Moroder, L.; Turk, D. Biochem. J 2004, 381, 511-517.

[9] (a) Murata, M.; Miyashita, S.; Yokoo, C.; Tamai, M.;Hanada, K.; Hatayama, K.; Towatari, T.; Nikawa, T.; Katunuma, N. FEBS Letters 1991, 280, 307-310. (b) Towatari, T.; Nikawa, T.; Murata, M.; Yokoo, C.; Tamai, M.; Hanada, K.; Katunuma, N. FEBS Letters 1991, 280, 311-315.

[10] For example, see: (a) Huang, Z.; McGowan, E. B.; Detwiler, T. C. J Med. Chem. 1992, 35, 2048-2054. (b) Gour-Salin, B. J.; Lachance, P.; Plouffe, C.; Storer, A.C.; Ménard, R. J. Med. Chem. 1993, 36, 720-725. (c) Schaschke, N.; Assfalg-Machleidt, I.; Machleidt, W.; Turk, D.; Moroder, L. Bioorg. Med. Chem. 1997, 5, 1789-1797. (d) Schaschke, N.; Assfalg-Machleidt, I.; Machleidt, W.; Moroder, L. FEBS Letters 1998, 421, 80-82. (e) Katunuma, N.; Murata, E.; Kakegawa, H.; Matsui, A.; Tsuzuki, H.; Tsuge, H.; Turk, D.; Turk, V.; Fukushima, M.; Tada, Y.; Asao, T. FEBS Letters 1999, 458, 6-10. (f) Schaschke, N.;Assfalg-Machleidt, I.; Machleidt, W.; Lassleben, T.; Sommerhoff, C. P.; Moroder, L. Bioorg. Med. Chem. Lett. 2000, 10, 677-680. (g) Schaschke, N.; Assfalg-Machleidt, I.; Lassleben, T.; Sommerhoff, C. P.; Moroder, L.; Machleidt, W. FEBS Letters 2000, 482, 91-96.

[11] (a) Tamai, M.; Yokoo, C.; Murata, M.; Oguma, K.; Sota, K.; Sato, E.; Kanaoka, Y. Chem. Pharm. Bull. 1987, 25,1098-1104.

[12] Bogyo, M.; Verhelst, S.; Bellingard-Dubouchaud, V.; Toba, S.; Greenbaum, D. Chem. Biol. 2000, 7, 27-38.

[13] Turk, D.; Podobnik, M.; Popovic, T.; Katunuma, N.; Bode, W.; Huber, R.; Turk, V. Biochemistry, 1995, 34, 4791-4797.

What is claimed is:

1. A method for synthesizing an epoxide peptide derivative, comprising:
(a) immobilizing a first peptide derivative to a solid support, whereby the first peptide derivative has a free amino group bound to a protective group;
(b) deprotecting the amino group of the immobilized compound of step (a);
(c) coupling the compound of step (b) to an diester epoxide to form an amide bond with the compound of step (b) to obtain an immobilized ester epoxide—first peptide derivative;
(d) converting the immobilized ester epoxide of step (c) to a free acid and coupling a second peptide derivative to the free acid on the support; and
(e) releasing the compound of step (d) from the solid support to yield an epoxide having two peptide derivatives attached thereto.

2. The method of claim 1 wherein the first and second peptide derivatives each comprise a peptoid.

3. The method of claim 1 wherein the epoxide having two peptide derivatives attached thereto of step (e) has a formula according to a compound selected from the group consisting of COMPOUND1, COMPOUND2, or COMPOUND 3, wherein AA₁, AA₂, R1, and R2 are amino acid side chains selected from naturally occurring amino acids, * is between 0 and 10, L1 is NH or O, and R and R3 are peptide derivatives, and R4 is hydrogen or a lower alkyl diamide.

4. The method of claim 1 wherein the two peptide derivatives each individually consist essentially of two amino acid derivatives.

5. The method of claim 1 wherein the solid support comprises a PEG-based Rink resin.

6. The method of claim 1 wherein the coupling of step (c) utilizes a reagent comprising nitrophenyl diester.

7. The method of claim 6 wherein the nitrophenyl diester is of the formula of compound 4.

8. The method of claim 5 wherein hydrolysis of the ester epoxide coupled to the first peptide is carried out in an alcohol solvent.

9. The method of claim 1 wherein the first peptide derivative and the second peptide derivative are comprised in a mixture of different peptide derivatives, each attached to a solid support, for generating a plurality of different epoxy peptide derivatives.

10. A method to synthesize epoxysuccinyl peptide derivatives, comprising:
(a) attaching a first amino acid derivative residue to a solid support;
(b) coupling a second amino acid derivative to the first amino acid derivative, while the first amino acid derivative is still on the solid support, to form a diamino acid complex;
(c) attaching an epoxy succinyl ester to an amine of the diamino acid complex on the solid support;
(d) hydrolyzing the epoxysuccinyl ester to form an epoxysuccinyl acid on the solid support; and
(e) eluting from the solid support the diamino acid-coupled dipeptide epoxysuccinyl acid.

11. The method of claim 10 wherein step (c) comprises the step of: capping an N-terminus of the diamino acid complex with an epoxysuccinyl synthon using an activated nitrophenyl ester.

12. The method of claim 10 wherein said solid support is a PEG-based Rink-resin.

13. The method of claim 10 further comprising the use of alcohol as a solvent system during ester bond cleavage.

14. The method of claim 10 further comprising the use of protected Fmoc-amino acid-amino acid—OtBu synthesized in solution as the dipeptide derivative of step (e).

15. The method of claim 14 wherein the protected Fmoc-amino acid-amino acid—OtBu is deprotected prior to coupling to the free acid on solid support.

16. The method of claim 15 further comprising the use of PyBOP as a coupling reagent for the dipeptide derivative of step (e).

17. The method of claim 10 further comprising the step of attaching a peptide derivative to the epoxysuccinyl synthon acid on the solid support prior to the step of eluting from the solid support.

18. The method of claim 10 wherein the first amino acid derivative and the second amino acid derivative are comprised in a mixture of different amino acid derivatives, each attached to a solid support, for generating a plurality of different epoxy peptide derivatives.

19. A compound useful in synthesizing epoxysuccinyl-based cysteine protease inhibitors, of the formula: 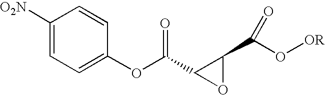
where R is a lower straight or branched chain alkyl group having one to ten carbon atoms.

20. An activity based probe for a protease of a formula selected from the group consisting of: 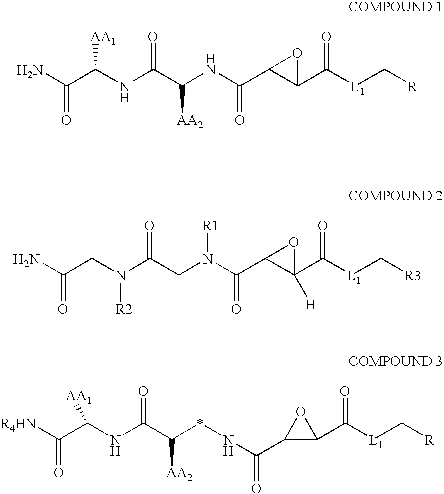
wherein AA₁, AA₂, R1 and R2 are each independently one of the twenty naturally occurring amino acid side chains; L₁ is NH or O; R and R3 are each a peptide derivative having from one to three amino acid residues; the compound is in the form of either an R,R or an S,S enantiomer; R4 is H or a lower alkyl diamide, and * is a carbon linker of 0-4 carbon atoms.

21. The compound of claim 20 wherein AA1 and AA2 are independently selected from: Gly, Thr, Ser, Trp, Tyr, Pro, His, Glu, Gln, Asp, Asn, Lys, and Arg.

22. The compound of claim 20 where L₁ is NH and R is a peptide having between 1 and three amino acid derivatives.

23. A compound of claim 20 having the formula of COMPOUND 1, wherein R is between 1 and three amino acid residues selected from one of the twenty naturally occurring amino acids.
